(19) United States
(12) Patent Application Publication
(10) Pub. No.: US 2009/0140071 A1
(43) Pub. Date: Jun. 4, 2009

(54) AIR FRESHENER ANTI-HUMIDITY TABLET

(75) Inventor: Eva SCHMIDT DIOS, LA CANONJA (Terragona) (ES)

Correspondence Address: DENNIS G. LAPOINTE;LAPOINTE LAW GROUP, PL, PO BOX 1294, TARPON SPRINGS, FL, 34688-1294, US

(73) Assignee: TOP GRADE, S.L., Terragona (ES)

(21) Appl. No.: 12/323,576

(22) Filed: Nov. 26, 2008

(30) Foreign Application Priority Data
Nov. 30, 2007 (EP) 07381073.1

(51) Int. Cl. A61L 9/04

(52) U.S. Cl. 239/34

(57) ABSTRACT

The present invention refers to an air freshener anti humidity tablet. The tablet comprises an agent which dries atmospheric humidity and is used in cases where it is also necessary to provide a pleasant fragrance. Its use is mainly appropriate for average sized closed rooms. The tablet which is the object of the invention comprises a drying material and is characterised by the fact that it is provided with at least one air freshener device which includes a container, part of the surface of which is in contact with the tablet and the other part in direct contact with the atmosphere, with the surface in contact with the tablet being impermeable and the surface in contact with the atmosphere being provided with a permeable membrane which realises a freshener agent from inside the container. In this way an anti-humidity function and an air freshener function are achieved using a means which also provides a gradual release of fragrance.

### RELATED APPLICATION

This application is a Paris Convention Application claiming priority from EP application 07381073.1 filed Nov. 30, 2007.

### FIELD OF THE INVENTION

The present invention refers to an air freshener anti-humidity tablet. The tablet comprises an agent for drying ambient humidity and, when necessary, is also used to absorb ambient humidity, to provide an agreeable aroma. It is designed mainly for use in closed environments of average dimensions such as, for example, rooms.

### OBJECT OF THE INVENTION

The tablet which is the object of the invention comprises a drying material and is characterised in that it is provided with at least one air freshener device which comprises a container which has part of its surface in contact with the tablet, and part of its surface in direct contact with the atmosphere, with the contact surface of the tablet being impermeable and the surface in contact with the atmosphere being provided with a permeable membrane which releases a freshening agent from inside the container.

In this way it is possible to combine in the same element an anti-humidity function and an air freshening function, with a means which provides a gradual release into the atmosphere of the fragrance.

### BACKGROUND OF THE INVENTION

Compacted means of humidity absorption using absorbent agents are well known, such as, for example those in the form of small granules or in larger tablets. These agents act by absorbing humidity and transforming it into water, which is then stored in a collector situated underneath. When the drying agent has been consumed, the liquid is emptied and a replacement is fitted.

Damp areas frequently generate bad odours. Therefore, some of the aforementioned humidity absorption or drying agents contain additives which release a pleasant fragrance, thus achieving a reduction in unpleasant odours.

However, these means include in their composition aromatic additives which have the disadvantage that their fragrance is rapidly released so that it is consumed before the absorption capacity of the anti-humidity has been exhausted.

Permeable membranes are also known which regulate the discharge over time of a liquid into the atmosphere.

The present invention resolves the previous disadvantage by means of a tablet which is provided with the means for controlling the rate at which the fragrance is released. In this way the rate of release of the vapours is considerably reduced, achieving an improved spread of the evaporation of the freshening agent throughout the durable life of the drying product.

### DESCRIPTION OF THE INVENTION

The present invention refers to a tablet which absorbs atmospheric humidity while at the same time providing a pleasant fragrance. The tablet includes a drying agent for the absorption of humidity. This agent could be, for example, calcium chloride which is compacted in tablet form and which may be given any shape.

Its basic application is the absorption of humidity and the freshener of small, medium or large sized closed areas.

The tablet which is the object of this invention is characterised in that it is provided with at least one air freshener device comprising a container which is provided with part of its surface in contact with the tablet and part of its surface in direct contact with the atmosphere, with the surface in contact with the atmosphere in possession of a permeable membrane which releases a fragrance agent in the container.

In this way both the drying and the freshener functions are combined, with the latter function carried out throughout the full useful life of the drying product. The tablet which is the object of this invention combines in a single element both functions through a means which provides a gradual release into the atmosphere of the fragrance, and which also enables the elimination of freshener additives from the composition of the anti-humidity tablet.

### DESCRIPTION OF THE DRAWINGS

The present descriptive report is accompanied by a drawing, which is illustrative of a preferred embodiment but which is in no way restrictive of the invention.

FIG. 1 shows a perspective diagram of a preferred embodiment of the invention.

### PREFERRED EMBODIMENT OF THE INVENTION

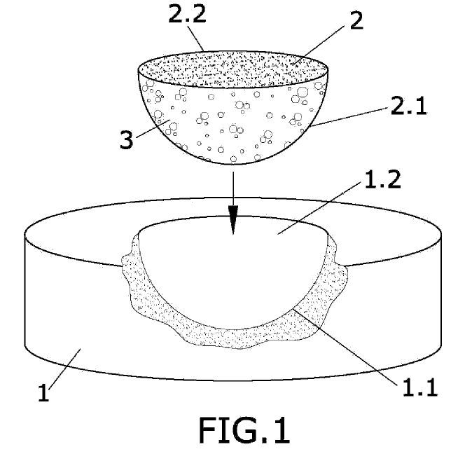

FIG. 1 shows a preferred embodiment of the tablet (1) which is the object of the invention.

The freshener device (2) is provided with impermeable walls 2.1) in contact with the tablet, so that the liquid content (3) of the freshener device (2) cannot migrate to the tablet, thus preventing any loss of the properties of the fragrance. The device (2) is provided with a wall, in this case in its upper part (2.2) which is in contact with the atmosphere. This wall (2.2) is provided with a permeable membrane which releases the freshener agent (3) through said membrane.

In the preferred embodiment shown, the table is provided with a cavity (1.1) which has the same shape as the wall (2.1) in contact with the tablet of the freshener element (2) and into which the tablet fits.

The freshener device (2) and the tablet (1) may be connected by various means, such as, for example, an adhesive.

1. Air freshener anti-humidity device comprising a drying material characterised in that it is provided with at least one freshener device (2) which includes a container having part of its surface (2.1) in contact with the tablet and part of its surface (2.2) in direct contact with the atmosphere and provided with a permeable membrane which releases a freshener agent (3) contained inside the container.

2. Air freshener anti-humidity device according to claim 1, characterised in that it is provided with a cavity (1.1) into which the freshening device (2) fits.

3. Air freshener anti-humidity device according to claim 2, characterised in that the freshener device (2) and the tablet (1) are connected by means of an adhesive.
